(19) United States
(12) Patent Application Publication
(10) Pub. No.: US 2009/0275671 A1
(43) Pub. Date: Nov. 5, 2009

(54) TWO COMPONENT BONE CEMENT COMPOSITION FOR VERTEBROPLASTY

(75) Inventors: Levinus Hendrik Koole, Gulpen (NL); Catharina Sibilla Josephine Van Hooy-Corstjens, Roosteren (NL); Tristan Laurens Bert Slots, Heythuysen (NL)

Correspondence Address: BANNER & WITCOFF, LTD., 28 STATE STREET, 28th FLOOR, BOSTON, MA, 02109-9601, US

(73) Assignee: Universiteit Maastricht, Maastricht (NL)

(21) Appl. No.: 12/432,421

(22) Filed: Apr. 29, 2009

Related U.S. Application Data
(63) Continuation of application No. PCT/NL2007/050521, filed on Oct. 31, 2007.

(30) Foreign Application Priority Data
Oct. 31, 2006 (EP) 06076954.4

(51) Int. Cl. A61K 47/32; C08L 33/10; A61L 24/06

(52) U.S. Cl. 514/772.4; 525/302; 523/117

(57) ABSTRACT

The invention is directed to an intrinsically radio-opaque two component bone cement, comprising a first component which contains at least one acrylate monomer and a second component which contains at least one initiator for the polymerisation of said acrylate monomer, wherein the at least one iodine containing radio-opacity providing polymer is present in at least one of the two components and wherein the cement, when mixed into a slurry has a viscosity, which is sufficiently low to allow injection of the cement slurry through a needle with a diameter in the range of 10-15 G used in percutaneous vertebroplasty.

### RELATED APPLICATIONS

This application is a continuation of PCT application no. PCT/NL2007/050521, designating the United States and filed Oct. 31, 2007; which claims the benefit of the filing date of European application no. 06076954.4 filed Oct. 31, 2006; each of which is hereby incorporated herein by reference in its entirety for all purposes.

### FIELD

The present invention relates to new radio-opaque bone cements for use in the treatment of vertebral compression fractures, resulting from osteoporosis, osteolytic metastases, myeloma or other causes. The cements pursuant to this invention feature intrinsic radio-opacity, and can be used in humans and in animals.

### BACKGROUND

Osteoporosis is a common disease that is characterised by structural deterioration of bone tissue. The disease leads to low bone mass, high bone fragility, and increased susceptibility to fractures. Osteoporosis-induced fractures occur mostly in the spine, with severe implications: collapse of the affected vertebral body (bodies), and loss of the physiological posture. Pain and reduced mobility are the most harassing consequences. Approximately 1,200,000 vertebral fractures, due to osteoporosis, occur annually in the US and Europe.

Percutaneous vertebroplasty is a minimally invasive technique in the treatment of vertebral compression fractures. The technique is used to augment and immobilise the affected vertebral body, and therefore to relieve pain and to restore the mobility and the quality of life of patients. Percutaneous vertebroplasty was first described by Galibert and Deramont in 1987. It has been developed into a cost-effective interventional procedure that can be performed under local anesthesia and conscious sedation as an outpatient procedure. The last years have shown a general acceptance of the technique, which reflects into rising numbers of patients and increased experience.

Important indications for percutaneous vertebroplasty are:
Painful primary and secondary osteoporotic vertebral body compression fracture
Painful vertebrae with extensive osteolysis or invasion secondary to benign or malign tumor
Painful vertebral fracture associated with osteonecrosis (Kummel disease)
   Important contraindications are:
Asymptomatic vertebral body compression fractures
Patient improving on medical therapy
Ongoing local or systemic infection
Allergy to bone cement or opacifying agent

Percutaneous vertebroplasty is essentially based on injection of a bone cement through a cannula, into the centre of the affected vertebral body. The cement hardens in situ, in a manner that is analogous to hardening of bone cements that are commonly used in arthroplasty procedures (e.g. total hip replacement, or total knee replacement). The exact positioning of the vertebroplasty cannula is important to assure satisfying filling of the vertebral body; this is accomplished through continuous anteroposterior and lateral X-ray fluoroscopic control. After correct positioning of the cannula, either unipedicular or bipedicular, the bone cement is prepared. During injection of the cement, continuous X-ray fluoroscopic observation is necessary to verify whether the vertebral body is completely filled with cement, and particularly to prevent excessive bone cement leakage. Patients are hospitalised at least for the day of the intervention.

The large majority of the percutaneous vertebroplasty procedures is based on the use of a poly(methylmethacrylate) (PMMA) bone cement. PMMA is by itself radiolucent (i.e. transparent for X-rays), i.e. addition of a contrast is mandatory. The contrast agent is, almost without exception, barium sulphate. A high concentration of barium sulphate will facilitate cement visualisation, but also has enormous drawbacks: it is known that barium sulphate can elicit a local inflammatory reaction, and high concentrations of barium sulphate render the cement slurry particularly viscous, which complicates the injection. Note that a viscous cement slurry requires a high pressure to flow through the cannula, which complicates the precision at which the cement can be deposited. Moreover, the biocompatibility of barium sulphate containing bone cements in the spine has not been investigated.

Technical problems with existing cements for vertebroplasty are associated with high viscosity and/or a level of radiopacity that is insufficient to adequately observe the injection process and ensure perfect location. Up to now, surgeons have sought their own solutions, as is evident from contemporary literature. For instance, X-ray contrast is routinely enhanced through addition of extra amounts of barium sulphate or metallic powders. Analogously, viscosity of the cement slurry is adapted routinely through varying the powder-to-liquid ratio during preparation of the cement. The clinical implications of these and other approaches are unclear.

Invariably, these modifications have led to an increase in viscosity, resulting in difficulties in the process of injection, such as difficulty in using the usual injection needles (i.e. those having a diameter in the range of 10-15 G), requiring larger forces and inhomogeneity of the composition, both possibly resulting in an inaccurate localisation of the bone cement.

Hence, there is an obvious need for controlled modifications of the formulation of acrylic bone cements for percutaneous vertebroplasty, which does not have the above disadvantages. In this respect it is to be noted that the conventional radio-opaque bone cements for use in knee and hip operations, are unsuitable for percutaneous vertebroplasty, as their radio-opacity is too low for the cement to be sufficiently visible.

A summary of the state of the art on the formulation of methacrylic bone cements for percutaneous vertebroplasty have appeared in the literature, such as in G. Lewis J. Biomed. Mater. Res. 2006, B76, 456-468, the contents of which is incorporated herein by way of reference.

### SUMMARY

The present invention has embodiments that provide solutions to one or more problems existing in the prior art with respect to percutaneous vertebroplasty, and the use of radio-opaque acrylic bone cements therein. One of the major problems in this area is that it has been difficult, thus far, to prepare acrylic bone cements for this specific application, in which a high level of radio-opacity is combined with favourable or optimal theological properties (flow characteristics) of the bone cement during the transient working phase in which the cement slurry is injected into a diseased or damaged vertebral body of the patient. While the dispersion of extra amounts of X-ray contrast agents (such as additional barium sulphate or metallic particles) may provide pragmatic solutions in the clinical practice, it is obvious that improved cement formulations are required.

This invention discloses that polymer biomaterials that show intrinsic radio-opacity provide an adequate and non-trivial solution to this problem. It is possible, on the basis of intrinsically radio-opaque polymer biomaterials, to combine a high level of X-ray contrast with a homogeneous slurry in the working phase; the slurry is free of aggregates and possesses rheological properties that allow easy and precise injection through narrow cannulae as are used in percutaneous vertebroplasty.

Moreover, the bone cement that is the subject of this invention is markedly non-toxic, presumable since there are no or less leachables. The counterpart bone cements may release toxic particles of the X-ray contrast agent (which is clearly the case for extensive filling with barium sulphate). Release of contrast agent in the vicinity of the bone cement may elicit an inflammatory response, possibly leading to osteolysis. These are highly unwanted consequences.

### DETAILED DESCRIPTION

The new bone cement of this invention has two major merits: (i), it has better flow characteristics in the slurry phase, thus allowing for more precise injections with lower risk of complications due to cement extravasation, and (ii), the material is non-toxic.

An essential component of the two-component bone cement of the invention is the presence of the intrinsically radio opaque iodine containing polymer, in at least one of the components thereof. This intrinsically radio opaque polymeric component can, in principle, be selected from all polymeric components that have an intrinsic radio opacity, i.e. that are visible in X-ray during and after injection into a diseased or broken vertebra.

Examples of suitable materials are polymeric components, preferably acrylate or methacrylate (co)polymers, bearing groups that impart the required high level of radio-opacity, by covalently bonded iodine groups. Preferably, the content of iodine or bromine in the polymeric material is such that the final bone cement contains at least 2 wt. % of the iodine that is covalently linked to a macromolecule. Most preferred amounts are between 2% and 20%.

The preferred materials are i.a. described in WO-A 96/05872, the contents of which is incorporated herein by way of reference.

In addition to the polymeric radio-opaque component one may also include an amount of polymer in the bone cement, which polymer is substantially not radio-opaque. Preferred polymers for this are methylmethacrylate homopolymers and copolymers, such such as methylmethacrylate-ethylacrylate copolymer, methylmethacrylate-methylacrylate copolymer, methylmethacrylate-butylmethacrylate copolymer or methylmethacrylate-styrene copolymer. The weight ratio of non radio-opaque to radio-opaque polymers is between 0 to 4, preferably between 0.25 and 4.

The bone cement of the invention is a two component system, preferably based on one liquid component and one powder component. In the aggregate of the two components, the bone cement comprises at least one acrylate monomer, preferably methylmethacrylate and/or butylmethacrylate, at least one initiator for the polymerisation of the acrylate monomer, a accelerator for the initiator, a radio-opacity providing polymer and optionally one or more of the components selected from the group of non-polymeric opacity agents, non or slightly radio-opaque polymers and usual additives for bone cements, such as antibiotics. The radio-opacity is provided by the intrinsically radio-opaque polymer, i.e. a polymer that has a chemical structure that provides radio-opacity through the presence of iodine atoms covalently linked to the polymer structure.

It is to be noted that the radio-opacity can optionally be enhanced by the presence of additional radio-opaque additives, such as barium sulphate or zirconium dioxide. These amounts should be less than the amount of the polymeric radio-opaque component, i.e. less than half of the radio-opacity will generally be generated by the additional additive. It is preferred that the radio-opacity is completely provided by the iodine containing polymer, but minor amounts of these other compounds may be used, as the leaching will anyway be substantially less than in conventional systems.

It is essential that the initiator for the polymerisation of the acrylate monomer and the acrylate monomer are not in the same component. Generally the initiator is in the powder component, preferably together with the intrinsically radio-opaque polymeric component, whereas the acrylate monomer forms the basis of the liquid component.

Suitable initiators are peroxides, such as benzoyl-peroxide.

As accelerator for the polymerisation, one can suitable use one can suitably use N,N-dimethyl-p-toluidine or 2-[4-(dimethylamino)phenyl]ethanol.

In one aspect, the present invention provides a bone cement that is based on the polymerisation of methacrylate monomers in situ, that is after injection of a cement slurry that is formed after mixing of a liquid component and a powder component. The mechanism of the hardening of the bone cement that is the subject of this invention is in close analogy to the mechanism of the hardening of existing bone cements that are already in clinical use in applications such as joint arthroplasty (e.g., replacement of the hip joint or of the knee joint), or percutaneous vertebroplasty.

The invention is thus defined as an intrinsically radio-opaque two component bone cement, comprising a first component which contains at least one acrylate monomer and a second component which contains at least one initiator for the polymerisation of said acrylate monomer, wherein the at least one radio-opacity providing polymer is present in at least one of the two components.

The bone cement of the present invention is preferably based on the same principle as the conventional bone cements, namely a two component system, comprising one liquid component and one powder component. Prior to injection these two components are mixed together in the required ratio and injected into the broken or diseased vertebra through a needle. The powder component preferably contains the intrinsically radio-opaque polymer.

The bone cement that is the subject of this invention hardens in three phases. The first phase is the mixing phase, in which the liquid component and the powder component are physically mixed. The mixing can occur manually with a spatula, or with the help of a bone cement mixing device. The second phase is the working phase. In the second phase, the bone cement is a slurry with a certain viscosity, which stays approximately constant during several minutes. During this second phase, the surgeon injects the bone cement into the vertebral body that requires augmentation. The third phase is the hardening phase; in this phase the polymerisation reaction proceeds in situ. This polymerisation reaction transforms the cement slurry into a hard material, through chemical conversion of the methacrylate reactive monomers that originate from the liquid component of the bone cement. During the third phase, the temperature of the cement rises. The three phases of the hardening of the bone cement that is the subject of this invention are in close analogy to the three phases of hardening of existing bone cements that are already in clinical use in applications such as joint arthroplasty (e.g., replacement of the hip joint or of the knee joint), or percutaneous vertebroplasty.

Surprisingly, the bone cement of the invention, through the use of the intrinsically radio-opaque polymer, i.e. the polymer providing the major amount or all of the radio-opacity, provides a combination of on the one hand, the high level of radio-opacity that is essential for percutaneous vertebroplasty, and on the other hand the relatively low viscosity that is needed for a proper injection into the vertebra.

In another aspect, the bone cement that is the subject of this invention requires the use of reactive ingredients during the preparation of the bone cement. These ingredients are selected in close analogy to the ingredients of existing bone cements that are already in clinical use in applications such as joint arthroplasty (e.g., replacement of the hip joint or of the knee joint), or percutaneous vertebroplasty.

Another aspect of the invention is that the bone cement is radio-opaque, i.e. capable of absorbing X-radiation. This property is essential with respect to the application of the bone cement, which is in the field of percutaneous vertebroplasty. It is essential to this invention that the radio-opacity of the bone cement that is the subject of this invention is an intrinsic property of the bone cement material. The radio-opacity of the bone cement that is the subject of this invention is not, or not exclusively, based on the presence of a radio-opaque additive.

The radio-opacity of the bone cement that is the subject of this invention originates from iodine atoms that are covalently linked to the macromolecules of the bone cement. The bone cement that is the subject of this invention is, therefore, intrinsically radio-opaque.

The present invention is that the present intrinsically radio-opaque bone cement opens the possibility to combine a high level of radio-opacity, which corresponds to clear visibility of the flowing bone cement slurry, on one hand, with exactly controllable viscosity of the bone cement slurry during the working phase of the bone cement curing. The combination of high radio-opacity and controllable viscosity is an essential aspect of the bone cement that is the subject of this invention, with regard to bone cements for percutaneous vertebroplasty that are already available in the market, or that have been described in the open scientific literature or in the open patent literature. All existing bone cements for percutaneous vertebroplasty derive their radio-opacity from the presence of a contrast agent, which is usually barium sulphate, or a combination of barium sulphate and metallic particles. These contrast agents have a clear tendency to form aggregates in the cement slurry, which is based on the lack of thermodynamic miscibility of the contrast agent and the polymer slurry that exists during the working phase of the bone cement hardening.

The formation of the aggregates proceeds without any control. The presence of the aggregates has a pronounced increasing effect on the viscosity of the bone cement slurry. If too viscous, injection of the bone cement slurry through the cannula is difficult, requiring high pressure. This is an important drawback that has a negative impact on the clinical success rate of the vertebroplasty operation. The advantage of the intrinsically radio-opaque bone cement that is the subject of this invention is that no aggregates are formed during the second phase of hardening of the bone cement preparation. This means that the bone cement of the invention can easily be injected through a needle having a 10-15 G (Gauge Number)

Therefore, it is possible to control exactly the level of the viscosity of the bone cement slurry, irrespective of the level of radio-opacity of the biomaterial. This feature is an influential advantage of the radio-opaque bone cement that is the subject of this invention, over all existing radio-opaque bone cements for percutaneous vertebroplasty. The possibility to control exactly the level of the viscosity of the bone cement slurry during the second phase of the bone cement hardening, irrespective of the level of radio-opacity of the biomaterial is a unique property of the bone cement that is the subject of this invention, that translates into higher success rates of clinical percutaneous vertebroplasty operations.

Another important advantage of the radio-opaque bone cement that is the subject of this invention, is the homogenous nature in which the X-ray contrast agent is dispersed. This implies that there is no leakage of any contrast agent in the body. This is in sharp contrast with existing radio-opaque bone cements for percutaneous vertebroplasty. It is known in the art that barium sulphate can leach from bone cement over prolonged time in situ. Free barium sulphate is toxic to bone cells and bone tissue, and may therefore cause osteolysis, which is highly undesirable loss of bone mass in the vicinity of the bone cement. The radio-opaque bone cement does not show any cytoxic effect in vitro and in vivo, which is a clear advantage over radio-opaque bone cements that are already in clinical use in applications such as joint arthroplasty (e.g., replacement of the hip joint or of the knee joint), or percutaneous vertebroplasty.

The preparation of the radio-opaque polymeric component of bone cement that is the subject of this invention can be performed via different routes. One of the preferred routes is based on the use of reactive methacrylate monomers such as, but not limited to, 2-[4-iodobenzoyl]-oxo-ethylmethacrylate. This structure combines the presence of (i), covalently bound iodine, in such a way that the carbon-iodine covalent bond is strong (i.e., not susceptible to heterolysis or homolysis through attack of a nucleophilic agent), and (ii), a reactive methacrylate group. It is known that 2-[4-iodobenzoyl]-oxo-ethylmethacrylate readily reacts with methyl methacrylate to form a random-type copolymer with excellent thermal and biological stability and an exceptionally high level of biocompatibility which is comparable to PMMA. The structure of 2-[4-iodobenzoyl]-oxo-ethylmethacrylate serves as an example, see FIG. 1. There are many more reactive monomers that contain one or more covalently bound iodine atoms on one hand, and one or more methacrylate groups. These can be found in the prior art. Homopolymers of such reactive iodine-containing monomers, and/or copolymers of such reactive iodine-containing monomers with methylmethacrylate and or butylmethacrylate are used as a component of the powder part of the bone cement that is the subject of this invention.

Another method to prepare the polymeric radio-opaque component of the bone cement that is the subject of this invention is to use polymers or copolymers that contain functional groups, such as hydroxyl groups or amino groups. Examples of such polymers of copolymers are poly-(2-hydroxyethylmethacrylate), poly(acrylamide), or copolymers of MMA and 2-hydroxyethylmethacrylate. Such polymers of copolymers can be reacted with iodine, iodine, or compounds that contain covalently linked iodine, such as—but not limited to—4-iodobenzoyl chloride or 4-iodobenzoic acid. In this manner, iodine is covalently attached to the polymer or copolymers, via newly generated ester bonds or amide bonds. These modified polymers or copolymers can be used as a component of the liquid part or the powder part of the radio-opaque bone cement.

Through use of the radio-opaque polymers or copolymers, prepared according to on of the methods described above, new intrinsically radio-opaque bone cements can be obtained in a straightforward manner. These materials are homogeneous and non-toxic which distinguishes them from existing radio-opaque bone cements. It is important to this invention that this distinction translates into markedly different rheological properties of the bone cement slurry that is generated during the second phase of bone cement hardening. This feature is non-trivial and provides an enormous advantage with regard to the intended application which is in percutaneous vertebroplastry. The radio-opaque bone cement that is the subject of this invention uniquely allows the combination of a high level of radio-opacity and desirable flow characteristics of the bone cement slurry. This combination is extremely difficult to achieve with formulations that are known art in the field of percutaneous vertebroplasty bone cements.

### DESCRIPTION OF FIGURES

FIG. 1 shows the structural formula of the iodine-containing methacrylate monomer 2-[4-iodobenzoyl]-oxo-ethylmethacrylate.

FIG. 2 shows X-ray contrast images of the cements A, B, C, D and E of the second series (powder-to-liquid ratio 0.6) of the subsequent examples. Note that cement E is practically invisible.

FIG. 3 shows scanning-electron micrographs at three different magnifications of cement A (backscatter mode). Note the presence of relatively large clumps of barium sulphate (white spots). These account for leakage of barium sulphate and inferior physical mechanical properties.

FIG. 4 shows scanning-electron micrographs at three different magnifications of cement B (backscatter mode). Dark circles result from the PMMA microspheres, and relatively light circles result from iodine-containing microspheres. The continuous phase is relatively light due to dissolution of relatively small iodine-containing microspheres during the mixing phase. Note the more homogeneous nature of the material, relative to cement A (FIG. 2).

FIG. 5 shows scanning-electron micrographs at three different magnifications of cement D (backscatter mode). Dark circles (PMMA) and light circles (iodine containing biomaterial), as well as barium sulphate clumps are clearly visible.

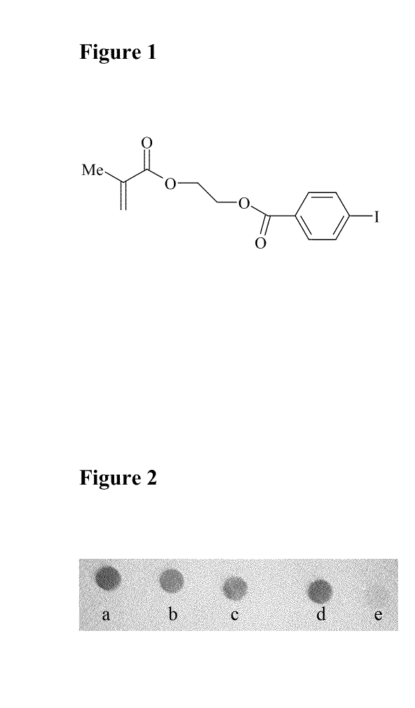

### EXAMPLE

### Preparation of PMMA

PMMA microsheres were prepared by suspension polymerisation. A mixture of 1 liter water containing poly(vinyl alcohol), poly(N-vinylpyrrolidone) and polyethylene glycol was taken into a reaction vessel and heated to 70° C. for 1 h under continuous stirring. A mixture of MMA (140 g) and a defined amount of radical initiator (benzoyl peroxide) was added with stirring. The reaction was continued for 3 h. Then, stirring was stopped and the vessel was cooled to ambient temperature. PMMA microspheres settled to the bottom of the reaction vessel. All microspheres were passed through a 200 μm sieve; particles that did not pass the sieve were discarded.

### Preparation of Iodine Containing Polymers

The monomer 2-[4-iodobenzoyl]-oxo-ethylmethacrylate was prepared according to a literature procedure. Microspheres were prepared according to the procedure described above for PMMA. Microspheres from both poly(2-[4-iodobenzoyl]-oxo-ethylmethacrylate-MMA) (copolymer 1:1 by mass), and poly (2-[4-iodobenzoyl]-oxo-ethylmethacrylate) (homopolymer) were prepared. All microspheres were passed through a 200 μm sieve; particles that did not pass the sieve were discarded.

Five different cements were prepared as is shown in Table 2.

TABLE 2
Compositions of experimental bone cements

| | PMMA | BaSO4 | I-copolymer | I-homopolymer |
|---|---|---|---|---|
| Cement | Mass % | Mass % | Mass % | Mass % |
| A | 70 | 30 | — | — |
| B | 40 | — | 60 | — |
| C | 70 | — | — | 30 |
| D | 55 | 15 | 30 | — |
| E | 100 | — | — | — |

First, to verify the influence of adding more radio-opacifier, cements were prepared with a liquid-to-powder ratio of 0.4, as is common for hip and knee arthroplasty bone cements. Compression tests, X-ray fluoroscopy, electron microscopy and cell compatibility tests were performed. Viscosities of the cement slurries were evaluated qualitatively.

Secondly, cements A, B, C, D and E were made with a liquid-to-powder ratio of 0.6, to create a low-viscosity bone cement as is desired for the intended percutaneous vertebroplasty application. Compression tests, X-ray fluoroscopy, electron microscopy and cell compatibility tests were performed. Viscosities of the cement slurries were evaluated qualitatively.

For the first series (i.e., liquid-to-powder ratio 0.4), mixing of the powder and liquid component yielded viscous slurries or dough-like mixtures in cases A through E. The viscosity was too high in each case; this precluded injection through a cannula as normally used in percutaneous vertebroplasty.

For the second series (i.e., liquid-to-powder ratio 0.6), the resulting cement slurries B, D and E had excellent viscosity properties; they were injectable through 10-15 Gauge cannula needles.

FIG. 2 shows the X-ray contrast of the different cements in the second series (after hardening). Cement B was slightly less radio-opaque as compared to A and D, but clearly more radio-opaque than the same cement with 10% BaSO₄ (by mass).

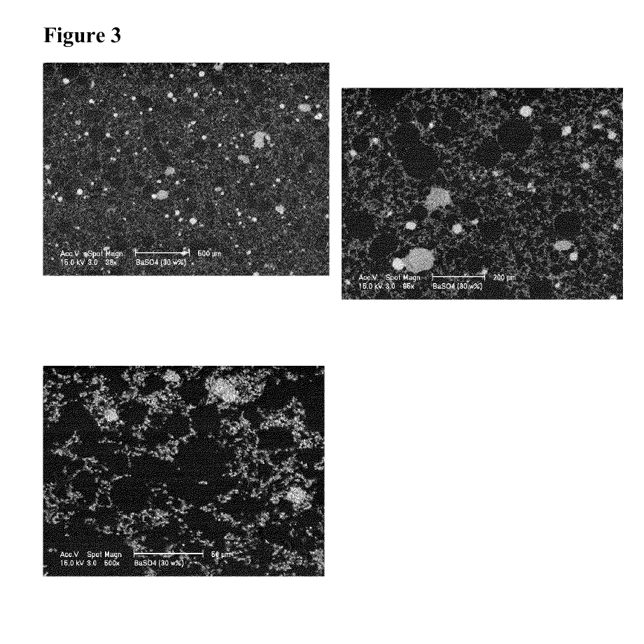

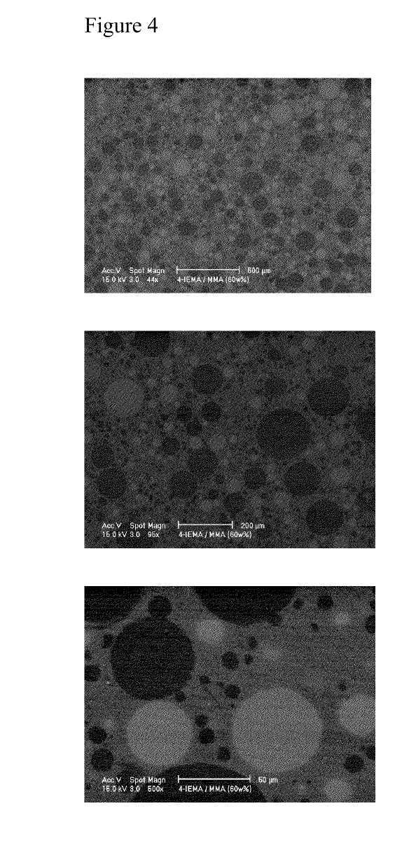

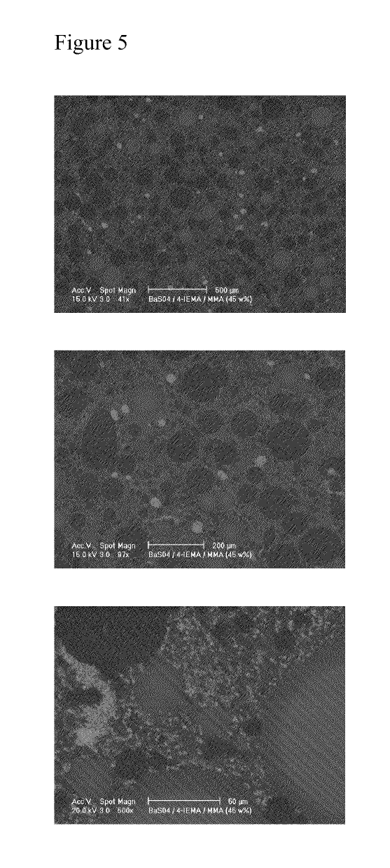

FIGS. 3 through 5 show the morphology of the cements in series 2. Note that large barium sulphate clumps are seen in FIG. 3 (SEM of cement A). FIG. 4 reveals the homogeneous nature of cement B, which derives its radio-opacity exclusively from the microspheres that consist of the copolymer poly(2-[4-iodobenzoyl]-oxo-ethylmethacrylate-MMA) (copolymer 1:1 by mass). FIG. 5 shows the morphology of cement D, which reveals the presence of barium sulphate clumps, albeit in lower amounts and smaller sizes as compared to FIG. 3.

The material properties of the cements A, B, D and E are summarised in Table 3.

TABLE 3
Compilation of material properties of the experimental
bone cements in series 2 (liquid-to-powder ratio 0.6)

| | | E-modulus | Yield stress |
|---|---|---|---|
| Cement | Contrast agent in powder | (GPa) | (MPa) |
| A | Barium sulphate | 1.6 | 60.5 +/− 3.9 |
| B | poly(2-[4-iodobenzoyl]-oxo- | 1.7 | 57.6 +/− 5.9 |
| | ethylmethacrylate-MMA) | | |
| D | BaSO4 and poly(2-[4-iodobenzoyl]- | 1.7 | 64.2 +/− 3.8 |
| | oxo-ethylmethacrylate-MMA) | | |
| E | None | 1.7 | 66.7 +/− 1.8 |

Investigation of the curing properties of the cements A, B, D, and E in the second series showed that the maximum temperature reached during the polymerisation is the same in all four cases; the maximum temperature is approximately 90° C. under laboratory conditions. It was found that the length of the second phase of the hardening process (working phase) was dependent on the concentration of the radical initiator, benzoyl peroxide. In all four cases, the concentration of benzoyl peroxide could be chosen in such a manner, that a working time of 10 minutes was achieved; this is the desired working time for the intended application in percutaneous vertebroplasty.

Using the MTT assay for cytotoxicity of extracts of the cements A, B, D and E, it was found that the cell viability in the extract of cement B was almost 100%, whereas an inhibition of the cell viability was noted for cements A and D. It was noted that the cytotoxicity effect increases with increasing amount of barium sulfate present. The scientific literature provides examples that the presence of barium sulphate particles influences the biological behaviour of bone cements. The presence of barium sulphate particles also intensifies the inflammatory response to PMMA debris. These effects must be expected to become more severe as the content of barium sulphate increases. It was described in the recent scientific literature that that PMMA bone cement with 30% barium sulphate, injected in the vertebrae of sheep, leads to an inflammatory response as is evident from the presence of foreign-body giant cells, whereas this is much less the case in the case of 10% barium sulphate.

The experimental data that are presented in this example reveal uniquely that the important physical mechanical properties of hardened bone cements, like compression modulus and yield stress, are by no means influenced in a negative sense, by using the copolymer poly((2-[4-iodobenzoyl]-oxo-ethylmethacrylate)-MMA) as the radio-opacifier. The advantages of doing this is are at least three-fold: (i), the cement is much more homogeneous as compared to cements with the same level of radio-opacity on the basis of a contrast additive; (ii), the cement slurry, present during the working phase is less viscous and has better theological properties (flow characteristics) and hence a better injectability; (iii) the cement is non-toxic, which is extremely important in the intended application (vertebroplasty) in which the cement is deposited close to the nerve bundles in the spine.

The implication from these findings is that bone cements with intrinsic radio-opacity have suitable properties for use in percutaneous vertebroplasty; they outperform existing bone cements for this purpose in several essential respects.

1. Intrinsically radio-opaque two component bone cement, comprising a first component which contains at least one acrylate monomer and a second component which contains at least one initiator for the polymerisation of said acrylate monomer, wherein at least one iodine containing radio-opacity providing polymer is present in at least one of the two components and wherein the cement, when mixed into a slurry has a viscosity, which is sufficiently low to allow injection of the cement slurry through a needle with a diameter in the range of 10-15 Gauge number used in percutaneous vertebroplasty.

2. Bone cement according to claim 1, wherein a polymer or copolymer containing methacrylate monomers that contain covalently linked iodine are used.

3. Bone cement according to claim 2, wherein the said monomer containing iodine is selected from the group of acrylates, ethacrylates or propylacrylates.

4. Bone cement according to claim 1, which comprises, in the aggregate of the two components, at least one acrylate monomer, at least one initiator for the polymerisation of the acrylate monomer, an accelerator for the initiator, the radio-opacity providing polymer and optionally one or more of the components selected from the group of non-polymeric opacity agents, non or slightly radio-opaque polymers and additives for bone cements.

5. Bone cement according to claim 1, wherein the bone cement contains barium sulfate, zirconium dioxide or another inorganic contrast agent.

6. A method of making a bone cement for percutaneous vertebroplasty comprising combining a first component which contains at least one acrylate monomer and a second component which contains at least one initiator for the polymerisation of said acrylate monomer, wherein at least one iodine containing radio-opacity providing polymer is present in at least one of the two components.

7. A method for percutaneous vertebroplasty, comprising preparing a bone cement by combining a first component which contains at least one acrylate monomer and a second component which contains at least one initiator for the polymerisation of said acrylate monomer, wherein at least one iodine containing radio-opacity providing polymer is present in at least one of the two components, and injecting the bone cement into the vertebra to be treated.
